(19) United States
(12) Patent Application Publication
(10) Pub. No.: US 2022/0126082 A1
(43) Pub. Date: Apr. 28, 2022

(54) Device for Controlling Fluid Flow and Set for Peritoneal Dialysis Comprising Said Device

(71) Applicant: Fresenius Medical Care Deutschland GmbH, Bad Homburg (DE)

(72) Inventors: Gang Fu, Shanghai (CN); Wanding Deng, Shanghai (CN); Xuefeng He, Shanghai (CN)

(21) Appl. No.: 17/605,746

(22) PCT Filed: Apr. 25, 2019
(86) PCT No.: PCT/CN2019/084303

§ 371 Date: Oct. 22, 2021

(51) Int. Cl. A61M 39/22; A61M 1/28

(52) U.S. Cl. CPC A61M 39/223; A61M 2039/226; A61M 1/28

(57) ABSTRACT

A device for controlling a fluid flow comprises: a housing) with which at least a first connection port, a second connection port and a third connection port are fluidly communicated; a closure means disposed within the housing; and a rotatable actuation means configured to selectively open or close the first connection port, the second connection port and the third connection port by operating the closure means; wherein the actuation means is configured to have at least five rotation positions such that the device can achieve at least five respective different fluid flow states. Also disclosed is a set for a peritoneal dialysis comprising such a device. The device is particularly suitable to be used as a patient connector for a continuous ambulatory peritoneal dialysis (CAPD), which connector is particularly suitable for use in Point-of-Care (POC) CAPD to manage preparation of CAPD solution and use of the CAPD solution.

### CROSS-REFERENCE TO RELATED APPLICATIONS

The present application is the national stage entry of International Patent Application No. PCT/CN2019/084303, filed on Apr. 25, 2019, the disclosure of which is incorporated herein by reference.

### TECHNICAL FIELD

The present disclosure relates to a device for controlling a fluid flow, in particular a patient connector for a peritoneal dialysis, and a corresponding set for the peritoneal dialysis.

### BACKGROUND ART

A dialysis treatment is a procedure for removing toxic substances and metabolites normally removed by the kidneys, and for aiding in regulation of fluid and electrolyte balance.

The dialysis treatment may be carried out by various types of dialysis procedures, such as a hemodialysis and a peritoneal dialysis (PD). The peritoneal dialysis often includes a continuous ambulatory peritoneal dialysis (CAPD) and an automated peritoneal dialysis (APD).

For the CAPD, a connector is needed to fluidly connect a PD solution bag to a peritoneal catheter to transfer the PD solution from the PD solution bag into an abdominal cavity of the patient and also fluidly connect a drainage bag to the peritoneal catheter to discharge the spent PD solution from the abdominal cavity of the patient into the drainage bag. Moreover, with the development of the dialysis procedure, the PD solution can be prepared by diluting concentrates for example prefilled in the PD solution bag, which even can allow the patient to prepare conveniently the fresh dialysis solution at home. In this case, the connector also must be used to inject for example water for injection (WFI) from a WFI machine into the PD solution bag to prepare the fresh dialysis solution. Such a CAPD may be called as Point of Care (POC) CAPD.

Therefore, it is necessary to improve the known design to better manage preparation of the CAPD solution and use of the CAPD solution.

### SUMMARY OF THE DISCLOSURE

In view of the problems existing in the prior art, an object of the present disclosure is to provide a device for controlling a fluid flow, in particular a patient connector for a peritoneal dialysis, and a corresponding set for the peritoneal dialysis.

For achieving this object, according to a first aspect of the present disclosure, provided is a device for controlling a fluid flow, comprising: a housing with which at least a first connection port, a second connection port and a third connection port are fluidly communicated; a closure means disposed within the housing; and a rotatable actuation means configured to selectively open or close the first connection port, the second connection port and the third connection port by operating the closure means; wherein the actuation means is configured to have at least five rotation positions such that the device can achieve at least five respective different fluid flow states.

According to an optional embodiment of the present disclosure, the first connection port is configured to be adapted to be fluidly connected to a peritoneal catheter or to a filling means, in particular a water-for-injection machine, for providing at least one composition required to prepare a dialysis solution; the second connection port is configured to be adapted to be fluidly connected to a dialysis solution bag; and the third connection port is configured to be adapted to be fluidly connected to a drainage bag. The peritoneal catheter herein also may be a catheter extension.

According to an optional embodiment of the present disclosure, the actuation means comprises a handle outside of the housing and the closure means can be actuated by the handle to control the fluid flow.

According to an optional embodiment of the present disclosure, the closure means comprises a closure ring rotatably actuated by the handle, wherein the closure ring has a closure body configured to close at least one of the first connection port, the second connection port and the third connection port, and two openings formed in the closure body and configured to be able to be aligned simultaneously with any two of the first connection port, the second connection port and the third connection port to establish a flow path therebetween, so that the fluid flow can be controlled by rotating the closure ring.

According to an optional embodiment of the present disclosure, the closure means further comprises a linearly movable closure element for closing an inlet into the first connection port, for example an open end of the peritoneal catheter, and the actuation means further comprises a cam actuated by the handle, wherein the linearly movable closure element can be pressurized by the cam to close the inlet.

According to an optional embodiment of the present disclosure, the closure means comprises a first linearly movable closure element for closing the first connection port, a second linearly movable closure element for closing the second connection port and a third linearly movable closure element for closing the third connection port, and the actuation means further comprises a motion transfer mechanism for selectively transferring rotation of the handle into linear movements of the first, second and third linearly movable closure elements to control the fluid flow.

According to an optional embodiment of the present disclosure, the motion transfer mechanism comprises a guide rail with which the first, second and third linearly movable closure elements engage and which can be rotated by the handle.

According to an optional embodiment of the present disclosure, the actuation means further comprises a cam actuated by the handle, wherein the first linearly movable closure element can be pressurized by the cam to close an inlet into the first connection port, for example an open end of the peritoneal catheter.

According to an optional embodiment of the present disclosure, in a first rotation position of the at least five rotation positions, the first connection port is opened, the second connection port is opened and the third connection port is closed, such that the at least one composition is allowed to be filled into the dialysis solution bag from the filling means through the first connection port and the second connection port; in a second rotation position of the at least five rotation positions, the first connection port is opened, the second connection port is closed and the third connection port is opened, such that a waste fluid in an abdominal cavity of a patient is allowed to be drained to the drainage bag through the first connection port and the third connection port; in a third rotation position of the at least five rotation positions, the first connection port is closed, the second connection port is opened and the third connection port is opened, such that the dialysis solution in the dialysis solution bag is allowed to be transferred to the drainage bag through the second connection port and the third connection port for achieving a flush procedure; in a fourth rotation position of the at least five rotation positions, the first connection port is opened, the second connection port is opened and the third connection port is closed, such that the dialysis solution in the dialysis solution bag is allowed to be transferred to the abdominal cavity of the patient through the second connection port and the first connection port; and in a fifth rotation position of the at least five rotation positions, the first connection port is closed, the second connection port is closed and the third connection port is closed, such that the device is allowed to be separated from the peritoneal catheter.

According to an optional embodiment of the present disclosure, the device is configured as a disc patient connector for a continuous ambulatory peritoneal dialysis.

According to an optional embodiment of the present disclosure, the first connection port is configured as an anti-contamination adapter.

According to an optional embodiment of the present disclosure, the anti-contamination adapter comprises: a first coupling portion adapted to be fluidly connected with the filling means; a second coupling portion adapted to be fluidly connected with the peritoneal catheter and being connected with the first coupling portion by a first breakable point; and a covering portion configured to cover the second coupling portion in an anti-contamination manner and to be connected with a connection portion of the anti-contamination adapter outside of the second coupling portion by a second breakable point; wherein the second coupling portion can be exposed so as to be allowed for being connected to the peritoneal catheter only when the first breakable point and the second breakable point are broken.

According to an optional embodiment of the present disclosure, the covering portion is connected with the first coupling portion such that the covering portion together with the first coupling portion can be separated from the second coupling portion.

According to an optional embodiment of the present disclosure, the first connection port, the second connection port and the third connection port are integrated with the housing into one piece; and/or the device further comprises a cover for closing the housing, which cover is provided with marks indicating the five rotation positions; and/or the housing has a cylindrical shape.

According to a second aspect of the present disclosure, provided is a set for a peritoneal dialysis, wherein the set comprises the above device.

According to the present disclosure, the connector is particularly suitable for use in Point-of-Care (POC) CAPD to manage preparation of CAPD solution and use of the CAPD solution.

### BRIEF DESCRIPTION OF THE DRAWINGS

The present disclosure and advantages thereof will be further understood by reading the following detailed description of some preferred exemplary embodiments with reference to the drawings in which:

FIG. 1 shows a double bag CAPD set according to an exemplary embodiment of the present disclosure and a filling means fluidly connected with the double bag CAPD set.

FIG. 2-FIG. 6 show five rotation positions of a device for controlling a fluid flow respectively, wherein the device is embodied herein as a disc patient connector for a continuous ambulatory peritoneal dialysis.

FIG. 7 shows an exemplary embodiment of the connector provided with an anti-contamination adapter as a first connection port.

FIG. 8 shows the connector as shown in FIG. 7 when a second coupling portion is exposed.

### DETAILED DESCRIPTION OF PREFERRED EMBODIMENTS

Some exemplary embodiments of the present disclosure will be described hereinafter in more details with reference to the drawings to better understand the basic concept of the present disclosure.

Generally, the present disclosure discloses a device for controlling a fluid flow, comprising: a housing with which at least a first connection port, a second connection port and a third connection port are fluidly communicated; a closure means disposed within the housing; and a rotatable actuation means configured to selectively open or close the first connection port, the second connection port and the third connection port by operating the closure means; wherein the actuation means is configured to have at least five rotation positions such that the device can achieve at least five respective different fluid flow states.

The device is especially suitable for use in a peritoneal dialysis to control flow of some kinds of fluids, such as, a fresh dialysis solution, a spent dialysis solution and a filling fluid required to prepare the fresh dialysis solution.

To facilitate understanding of the present disclosure, use of the device in a peritoneal dialysis set will be described below only as an illustrative example.

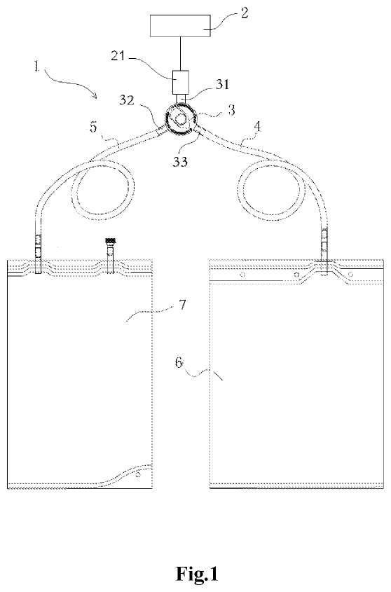

Firstly, a double bag CAPD set 1 according to an exemplary embodiment of the present disclosure will be described with reference to FIG. 1. In addition to the double bag CAPD set 1, FIG. 1 also schematically shows a filling means 2 fluidly connected with the double bag CAPD set 1.

As shown in FIG. 1, the double-bag CAPD set 1 mainly comprises the device described above, embodied herein as a connector 3 (here in a form of a disc connector), a drainage transfer tubing 4, a solution transfer tubing 5, a drainage bag 6, and a dialysis solution bag 7.

The connector 3 has a first connection port 31, a second connection port 32 acting as a dialysis solution port, and a third connection port 33 acting as a drainage connection port.

As also can be seen from FIG. 1, the first connection port 31 can be fluidly connected to the filling means 2 for example by means of a corresponding connector 21 of the filling means 2 to receive any required fluid, for example at least one composition required to prepare the fresh dialysis solution, in particular water for injection. Further, the first connection port 31 also can be fluidly connected to a peritoneal catheter/catheter extension (not shown) from a patient to infuse the fresh dialysis solution into an abdominal cavity of the patient or discharge the spent dialysis solution from the abdominal cavity of the patient. The second connection port 32 can be fluidly connected with the solution bag 7 by the solution transfer tubing 5, and the third connection port 33 can be fluidly connected with the drainage bag 6 by the drainage transfer tubing 4.

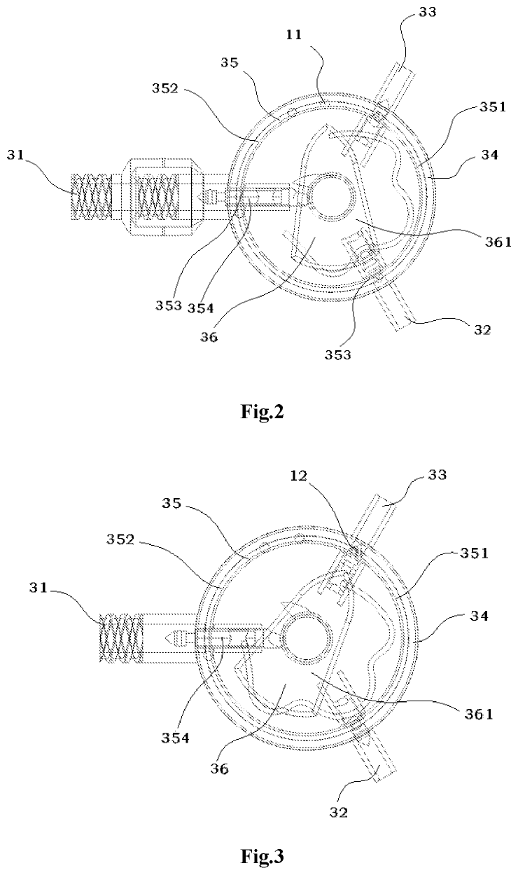

As described above, the actuation means is configured to have at least five rotation positions such that the device can achieve at least five respective different fluid flow states. According to one exemplary embodiment of the present disclosure, the connector 3 is configured to have the following five rotation positions as shown in FIG. 2-FIG. 6:

1) in a first rotation position 11 as shown in FIG. 2, the first connection port 31 is opened, the second connection port 32 is opened and the third connection port 33 is closed, such that the at least one composition is allowed to be filled into the dialysis solution bag 7 from the filling means 2, for example a water-for-injection machine through the first connection port 31 and the second connection port 32, thereby allowing to be mixed with other compositions prefilled in the dialysis solution bag 7 to prepare the fresh dialysis solution;

2) in a second rotation position 12 as shown in FIG. 3, the first connection port 31 is opened, the second connection port 32 is closed and the third connection port 33 is opened, such that a waste fluid, for example the spent dialysis solution in the abdominal cavity of the patient is allowed to be drained to the drainage bag 6 through the first connection port 31 and the third connection port 33;

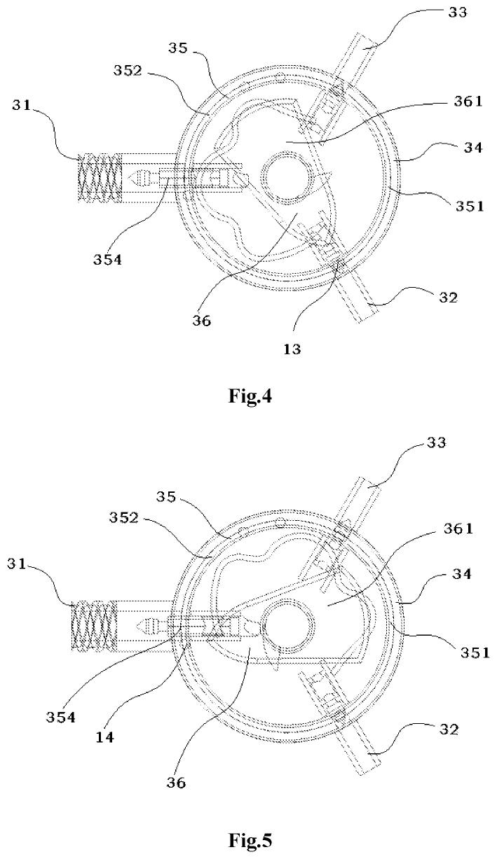

3) in a third rotation position 13 as shown in FIG. 4, the first connection port 31 is closed, the second connection port 32 is opened and the third connection port 33 is opened, such that the dialysis solution in the dialysis solution bag 7 is allowed to be transferred to the drainage bag 6 through the second connection port 32 and the third connection port 33 for achieving a flush procedure, thereby allowing to clean an interior of the connector 3;

4) in a fourth rotation position 14 as shown in FIG. 5, the first connection port 31 is opened, the second connection port 32 is opened and the third connection port 33 is closed, such that the dialysis solution in the dialysis solution bag 7 is allowed to be transferred to the abdominal cavity of the patient through the second connection port 32 and the first connection port 31, thereby allowing to execute the peritoneal dialysis; and

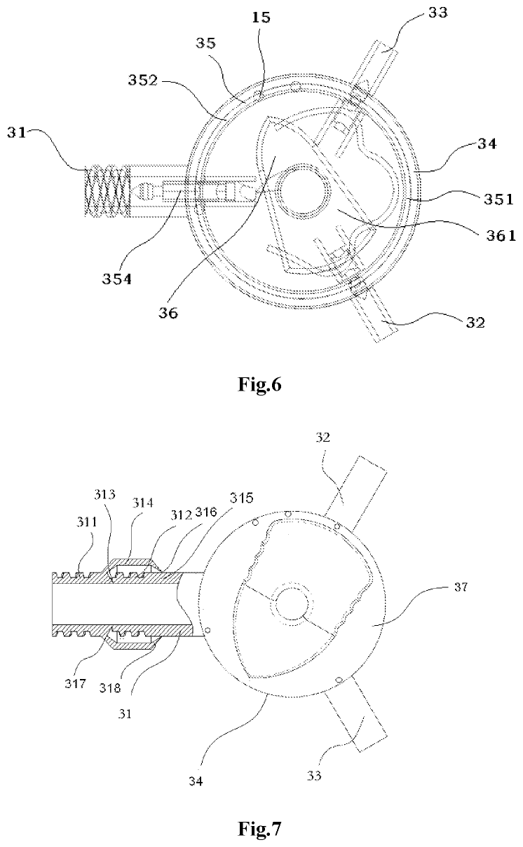

5) in a fifth rotation position 15 as shown in FIG. 6, the first connection port 31 is closed, the second connection port 32 is closed and the third connection port 33 is closed, such that the connector 3 is allowed to be separated from the peritoneal catheter.

Specifically, for the connector 3 as shown in FIG. 2-FIG. 6, it further comprises a housing 34 with which the first connection port 31, the second connection port 32 and the third connection port 33 are fluidly communicated; a closure means 35 disposed within the housing 34; and a rotatable actuation means 36 configured to selectively open or close the first connection port 31, the second connection port 32 and the third connection port 33 by operating the closure means 35. The closure means 35 can be actuated by the actuation means 36 to actually execute selective opening or closing of the first connection port 31, the second connection port 32 and the third connection port 33.

According to one exemplary embodiment of the present disclosure, the actuation means 36 may comprise a handle 361 outside of the housing 34 and the closure means 35 can be actuated by the handle 361 to control the fluid flow. As shown in FIG. 2-FIG. 6, when the handle 361 is rotated such that it's tip end points to different rotation positions, the connector 3 can achieve different fluid flow states.

According to one exemplary embodiment of the present disclosure, the closure means 35 comprises a closure ring 351 rotatably actuated by the handle 361, wherein the closure ring 351 may have a closure body 352 configured to close at least one of the first connection port 31, the second connection port 32 and the third connection port 33, and two openings 353 formed in the closure body 352 and configured to be able to be aligned simultaneously with any two of the first connection port 31, the second connection port 32 and the third connection port 33 to establish a flow path therebetween, so that the fluid flow can be controlled by rotating the closure ring 351.

Preferably, the housing 34 is configured in a cylindrical shape and the closure ring 351 is configured as a circular ring which can be disposed at an inner circumferential side of the housing 34.

For allowing the connector 3 to be separated safely from the peritoneal catheter without flowing of the dialysis solution infused into the abdominal cavity out of the abdominal cavity in the fifth rotation position 15 as shown in FIG. 6, the closure means 35 may further comprise a linearly movable closure element 354, preferably configured as a plug, for closing an inlet into the first connection port 31, for example an open end of the peritoneal catheter, and the actuation means 36 may further comprise a cam 362 actuated by the handle 361, wherein the linearly movable closure element 354 can be pressurized by the cam 362 to close the inlet. Preferably, the linearly movable closure element 354 is inserted into and maintained on the open end of the peritoneal catheter to block the open end when the connector 3 is separated from the peritoneal catheter. Obviously, the linearly movable closure element 354 also can help to more reliably close the first connection port 31, for example in the third rotation position 13 as shown in FIG. 4.

Of course, any suitable linearly movable closure elements also can be provided for the second connection port 32 and the third connection port 33 to help to close the second connection port 32 and the third connection port 33.

It may be understood by the skilled person in the art that the closure means and/or the actuation means is not limited to the above configurations. For example, according to another exemplary embodiment of the present disclosure, the closure means may comprise a first linearly movable closure element for closing the first connection port 31, a second linearly movable closure element for closing the second connection port 32 and a third linearly movable closure element for closing the third connection port 33, and the actuation means further comprises a motion transfer mechanism for selectively transferring rotation of the handle 361 into linear movements of the first, second and third linearly movable closure elements to control the fluid flow.

The motion transfer mechanism can be embodied in any suitable manners. For example, according to one exemplary embodiment of the present disclosure, the motion transfer mechanism comprises a guide rail with which the first, second and third linearly movable closure elements engage and which can be rotated by the handle 361. Preferably, the first, second and third linearly movable closure elements each are provided with a groove which engages with the guide rail, thereby selectively moving the first, second and third linearly movable closure elements toward or away from the first connection port 31, the second connection port 32 and the third connection port 33 when the guide rail is actuated by the handle 361.

According to one exemplary embodiment of the present disclosure, the first, second and/or third linearly movable closure element is configured to be movable radially toward or away from the first connection port 31, the second connection port 32 and/or the third connection port 33 respectively, which is very advantageous when the first connection port 31, the second connection port 32 and/or the third connection port 33 is disposed radially.

Preferably, for each of the first, second and third linearly movable closure elements, a guide structure, for example a guide channel is provided to ensure its linear movement.

Similarly, according to one exemplary embodiment of the present disclosure, the actuation means may further comprise a cam actuated by the handle 361, wherein the first linearly movable closure element can be pressurized by the cam to close an inlet into the first connection port 31, for example an open end of the peritoneal catheter.

As described above, when the fresh dialysis solution is prepared by for example diluting concentrates prefilled in the dialysis solution bag 7, the first connection port 31 is firstly connected to the filling means 2 to fill the required composition from the filling means 2 into the dialysis solution bag 7 to prepare the fresh dialysis solution in the first rotation position as shown in FIG. 2, and then is connected to the peritoneal catheter at least in the fourth rotation position as shown in FIG. 5 to transfer the prepared fresh dialysis solution to the abdominal cavity of the patient. Therefore, there is a possible crossing infection for the patient at the first connection port 31.

For avoiding such a crossing infection, according to one exemplary embodiment of the present disclosure, the first connection port 31 is configured as an anti-contamination adapter. In fact, FIG. 2 shows such an anti-contamination adapter.

However, for more clarity, FIG. 7 shows an exemplary embodiment of the connector 3 provided with the anti-contamination adapter as the first connection port 31, wherein the first connection port 31 is shown in a partial section view.

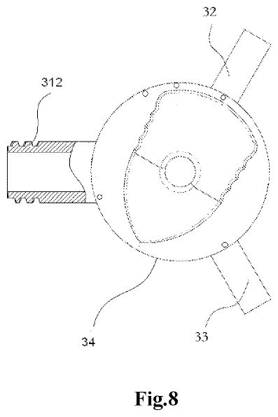

As shown in FIG. 7, the anti-contamination adapter comprises: a first coupling portion 311 adapted to be fluidly connected with the filling means 2; a second coupling portion 312 adapted to be fluidly connected with the peritoneal catheter and being connected with the first coupling portion 311 by a first breakable point 313; and a covering portion 314 configured to cover the second coupling portion 312 in an anti-contamination manner and to be connected with a connection portion 315 of the anti-contamination adapter outside the second coupling portion 312 by a second breakable point 316; wherein the second coupling portion 312 can be exposed so as to be allowed for being connected to the peritoneal catheter only when the first breakable point 313 and the second breakable point 316 are broken, as shown in FIG. 8. Therefore, the crossing infection can be avoided by using the anti-contamination adapter.

According to an exemplary embodiment of the present disclosure, the covering portion 314 is connected with the first coupling portion 311 such that the covering portion 314 together with the first coupling portion 311 can be separated from the second coupling portion 312.

According to an exemplary embodiment of the present disclosure, the first connection port 31, the second connection port 32 and the third connection port 33 are integrated with the housing 34 into one piece.

According to an exemplary embodiment of the present disclosure, the connector 3 further comprises a cover 37 for closing the housing 34, which cover is provided with marks indicating the five rotation positions.

While certain embodiments have been described, these embodiments have been presented by way of example only, and are not intended to limit the scope of the present disclosure. The attached claims and their equivalents are intended to cover all the modifications, substitutions and changes as would fall within the scope and spirit of the present disclosure.

1-15. (canceled)

16. A device for controlling a fluid flow, comprising:
a housing with which at least a first connection port, a second connection port and a third connection port are fluidly communicated;
a closure means disposed within the housing; and
a rotatable actuation means configured to selectively open or close the first connection port, the second connection port and the third connection port by operating the closure means,
wherein the actuation means is configured to have at least five rotation positions such that the device can achieve at least five respective different fluid flow states.

17. The device according to claim 16, wherein:
the first connection port is configured to be adapted to be fluidly connected to a peritoneal catheter or to a filling means, in particular a water-for-injection machine, for providing at least one composition required to prepare a dialysis solution;
the second connection port is configured to be adapted to be fluidly connected to a dialysis solution bag; and
the third connection port is configured to be adapted to be fluidly connected to a drainage bag.

18. The device according to claim 17, wherein the actuation means comprises a handle outside of the housing and the closure means can be actuated by the handle to control the fluid flow.

19. The device according to claim 18, wherein the closure means comprises a closure ring rotatably actuated by the handle, and
wherein the closure ring has: (i) a closure body configured to close at least one of the first connection port, the second connection port and the third connection port, and (ii) two openings formed in the closure body that are configured to be able to be aligned simultaneously with any two of the first connection port, the second connection port and the third connection port to establish a flow path therebetween so that the fluid flow can be controlled by rotating the closure ring.

20. The device according to claim 19, wherein the closure means further comprises a linearly movable closure element for closing an inlet into the first connection port, for example an open end of the peritoneal catheter, and
wherein the actuation means further comprises a cam actuated by the handle, wherein the linearly movable closure element can be pressurized by the cam to close the inlet.

21. The device according to claim 18, wherein the closure means comprises:
a first linearly movable closure element for closing the first connection port;
a second linearly movable closure element for closing the second connection port; and
a third linearly movable closure element for closing the third connection port, and
wherein the actuation means further comprises a motion transfer mechanism for selectively transferring rotation of the handle into linear movements of the first, second and third linearly movable closure elements to control the fluid flow.

22. The device according to claim 21, wherein the motion transfer mechanism comprises a guide rail with which the first, second and third linearly movable closure elements engage and which can be rotated by the handle.

23. The device according to claim 22, wherein the actuation means further comprises a cam actuated by the handle, wherein the first linearly movable closure element can be pressurized by the cam to close an inlet into the first connection port, for example an open end of the peritoneal catheter.

24. The device according to claim 17, wherein:
in a first rotation position of the at least five rotation positions, the first connection port is opened, the second connection port is opened and the third connection port is closed, such that the at least one composition is allowed to be filled into the dialysis solution bag from the filling means through the first connection port and the second connection port;
in a second rotation position of the at least five rotation positions, the first connection port is opened, the second connection port is closed and the third connection port is opened, such that a waste fluid in an abdominal cavity of a patient is allowed to be drained to the drainage bag through the first connection port and the third connection port;
in a third rotation position of the at least five rotation positions, the first connection port is closed, the second connection port is opened and the third connection port is opened, such that the dialysis solution in the dialysis solution bag is allowed to be transferred to the drainage bag through the second connection port and the third connection port for achieving a flush procedure;
in a fourth rotation position of the at least five rotation positions, the first connection port is opened, the second connection port is opened and the third connection port is closed, such that the dialysis solution in the dialysis solution bag is allowed to be transferred to the abdominal cavity of the patient through the second connection port and the first connection port; and
in a fifth rotation position of the at least five rotation positions, the first connection port is closed, the second connection port is closed and the third connection port is closed, such that the device is allowed to be separated from the peritoneal catheter.

25. The device according to claim 18, wherein:
in a first rotation position of the at least five rotation positions, the first connection port is opened, the second connection port is opened and the third connection port is closed, such that the at least one composition is allowed to be filled into the dialysis solution bag from the filling means through the first connection port and the second connection port;
in a second rotation position of the at least five rotation positions, the first connection port is opened, the second connection port is closed and the third connection port is opened, such that a waste fluid in an abdominal cavity of a patient is allowed to be drained to the drainage bag through the first connection port and the third connection port;
in a third rotation position of the at least five rotation positions, the first connection port is closed, the second connection port is opened and the third connection port is opened, such that the dialysis solution in the dialysis solution bag is allowed to be transferred to the drainage bag through the second connection port and the third connection port for achieving a flush procedure;
in a fourth rotation position of the at least five rotation positions, the first connection port is opened, the second connection port is opened and the third connection port is closed, such that the dialysis solution in the dialysis solution bag is allowed to be transferred to the abdominal cavity of the patient through the second connection port and the first connection port; and
in a fifth rotation position of the at least five rotation positions, the first connection port is closed, the second connection port is closed and the third connection port is closed, such that the device is allowed to be separated from the peritoneal catheter.

26. The device according to claim 19, wherein:
in a first rotation position of the at least five rotation positions, the first connection port is opened, the second connection port is opened and the third connection port is closed, such that the at least one composition is allowed to be filled into the dialysis solution bag from the filling means through the first connection port and the second connection port;
in a second rotation position of the at least five rotation positions, the first connection port is opened, the second connection port is closed and the third connection port is opened, such that a waste fluid in an abdominal cavity of a patient is allowed to be drained to the drainage bag through the first connection port and the third connection port;
in a third rotation position of the at least five rotation positions, the first connection port is closed, the second connection port is opened and the third connection port is opened, such that the dialysis solution in the dialysis solution bag is allowed to be transferred to the drainage bag through the second connection port and the third connection port for achieving a flush procedure;
in a fourth rotation position of the at least five rotation positions, the first connection port is opened, the second connection port is opened and the third connection port is closed, such that the dialysis solution in the dialysis solution bag is allowed to be transferred to the abdominal cavity of the patient through the second connection port and the first connection port; and
in a fifth rotation position of the at least five rotation positions, the first connection port is closed, the second connection port is closed and the third connection port is closed, such that the device is allowed to be separated from the peritoneal catheter.

27. The device according to claim 20, wherein:
in a first rotation position of the at least five rotation positions, the first connection port is opened, the second connection port is opened and the third connection port is closed, such that the at least one composition is allowed to be filled into the dialysis solution bag from the filling means through the first connection port and the second connection port;
in a second rotation position of the at least five rotation positions, the first connection port is opened, the second connection port is closed and the third connection port is opened, such that a waste fluid in an abdominal cavity of a patient is allowed to be drained to the drainage bag through the first connection port and the third connection port;
in a third rotation position of the at least five rotation positions, the first connection port is closed, the second connection port is opened and the third connection port is opened, such that the dialysis solution in the dialysis solution bag is allowed to be transferred to the drainage bag through the second connection port and the third connection port for achieving a flush procedure;
in a fourth rotation position of the at least five rotation positions, the first connection port is opened, the second connection port is opened and the third connection port is closed, such that the dialysis solution in the dialysis solution bag is allowed to be transferred to the abdominal cavity of the patient through the second connection port and the first connection port; and
in a fifth rotation position of the at least five rotation positions, the first connection port is closed, the second connection port is closed and the third connection port is closed, such that the device is allowed to be separated from the peritoneal catheter.

28. The device according to claim 21, wherein:
in a first rotation position of the at least five rotation positions, the first connection port is opened, the second connection port is opened and the third connection port is closed, such that the at least one composition is allowed to be filled into the dialysis solution bag from the filling means through the first connection port and the second connection port;
in a second rotation position of the at least five rotation positions, the first connection port is opened, the second connection port is closed and the third connection port is opened, such that a waste fluid in an abdominal cavity of a patient is allowed to be drained to the drainage bag through the first connection port and the third connection port;
in a third rotation position of the at least five rotation positions, the first connection port is closed, the second connection port is opened and the third connection port is opened, such that the dialysis solution in the dialysis solution bag is allowed to be transferred to the drainage bag through the second connection port and the third connection port for achieving a flush procedure;
in a fourth rotation position of the at least five rotation positions, the first connection port is opened, the second connection port is opened and the third connection port is closed, such that the dialysis solution in the dialysis solution bag is allowed to be transferred to the abdominal cavity of the patient through the second connection port and the first connection port; and
in a fifth rotation position of the at least five rotation positions, the first connection port is closed, the second connection port is closed and the third connection port is closed, such that the device is allowed to be separated from the peritoneal catheter.

29. The device according to claim 22, wherein:
in a first rotation position of the at least five rotation positions, the first connection port is opened, the second connection port is opened and the third connection port is closed, such that the at least one composition is allowed to be filled into the dialysis solution bag from the filling means through the first connection port and the second connection port;
in a second rotation position of the at least five rotation positions, the first connection port is opened, the second connection port is closed and the third connection port is opened, such that a waste fluid in an abdominal cavity of a patient is allowed to be drained to the drainage bag through the first connection port and the third connection port;
in a third rotation position of the at least five rotation positions, the first connection port is closed, the second connection port is opened and the third connection port is opened, such that the dialysis solution in the dialysis solution bag is allowed to be transferred to the drainage bag through the second connection port and the third connection port for achieving a flush procedure;
in a fourth rotation position of the at least five rotation positions, the first connection port is opened, the second connection port is opened and the third connection port is closed, such that the dialysis solution in the dialysis solution bag is allowed to be transferred to the abdominal cavity of the patient through the second connection port and the first connection port; and
in a fifth rotation position of the at least five rotation positions, the first connection port is closed, the second connection port is closed and the third connection port is closed, such that the device is allowed to be separated from the peritoneal catheter.

30. The device according to claim 16, wherein the device is configured as a disc patient connector for a continuous ambulatory peritoneal dialysis.

31. The device according to claim 17, wherein the first connection port is configured as an anti-contamination adapter.

32. The device according to claim 31, wherein the anti-contamination adapter comprises:
a first coupling portion adapted to be fluidly connected with the filling means;
a second coupling portion adapted to be fluidly connected with the peritoneal catheter and being connected with the first coupling portion by a first breakable point; and
a covering portion configured to cover the second coupling portion in an anti-contamination manner and to be connected with a connection portion of the anti-contamination adapter outside of the second coupling portion by a second breakable point,
wherein the second coupling portion can be exposed so as to be allowed for being connected to the peritoneal catheter only when the first breakable point and the second breakable point are broken.

33. The device according to claim 32, wherein the covering portion is connected with the first coupling portion such that the covering portion together with the first coupling portion can be separated from the second coupling portion.

34. The device according to claim 16, wherein:
the first connection port, the second connection port and the third connection port are integrated with the housing into one piece; and/or
the device further comprises a cover for closing the housing, which cover is provided with marks indicating the five rotation positions; and/or
the housing has a cylindrical shape.

35. A set for peritoneal dialysis, wherein the set comprises the device according to claim 16.
